(19) United States
(12) Patent Application Publication
(10) Pub. No.: US 2009/0220964 A1
(43) Pub. Date: Sep. 3, 2009

(54) METHODS FOR DIAGNOSING AND TREATING CANCERS VIA MANIPULATIONS OF A...PATHWAY

(75) Inventor: Maria F. Czyzk-Krzeska, Cincinnati, OH (US)

Correspondence Address: DINSMORE & SHOHL LLP, 1900 CHEMED CENTER, 255 EAST FIFTH STREET, CINCINNATI, OH, 45202, US

(73) Assignee: UNIVERSITY OF CINCINNATI, Cincinnati, OH (US)

(21) Appl. No.: 12/161,230

(22) PCT Filed: Jan. 18, 2007
(86) PCT No.: PCT/US07/01343

§ 371 Date: Nov. 7, 2008

Related U.S. Application Data
(60) Provisional application No. 60/759,838, filed on Jan. 18, 2006.

(51) Int. Cl. C12Q 1/68; G01N 33/573; C12N 5/02

(52) U.S. Cl. 435/6; 435/7.4; 435/375

(57) ABSTRACT

Methods for diagnosing, treating, and screening for cancer based on manipulations of a newly discovered pVHL dependent, non-degradative ubiquitylation pathway of Rpb1. In particular, methods comprising the use of biomarkers implicating the pathway, and promoters of either or both of P1465 hydroxylation and CTD Ser-5 phosphorylation of Rpb1. Specifically, the methods may be used to inhibit tumor growth, including, in particular, carcinomas such as renal clear cell carcinoma.

### RELATED APPLICATION

This Application claims priority under Title 35, United States Code, §119, to U.S. Provisional Application Ser. No. 60/759,838, filed on Jan. 18, 2006.

### FIELD OF THE INVENTION

The invention relates to methods for diagnosing and treating certain cancers by use of specified biomarkers and by manipulating various proteins and enzymes implicated in the newly discovered non-degradative pVHL-dependent Rpb1 ubiquitylation pathway. In particular, specific methods relate to treating carcinomas such as renal clear cell carcinoma. The invention further relates to methods of monitoring individuals at risk for developing certain cancers.

### BACKGROUND OF THE INVENTION

The most recognized function of the von Hippel-Lindau tumor suppressor protein (pVHL) is ubiquitylation of alpha subunits of hypoxia-inducible transcription factor, HIF-α, which targets them for proteasomal degradation. A key event enabling binding of pVHL to HIF-αs is O₂-regulated hydroxylation of proline residues within their oxygen dependent degradation domain (ODDD). The accumulation of HIF-α and resultant activation of HIF-responsive genes, including angiogenic factors, that occurs in response to the loss of pVHL activity are well recognized events stimulating growth of sporadic and familial renal tumors.

pVHL is a component of a multiprotein complex containing elongins C and 3, Cullin 2, and the RINGH2 finger protein Rbx-1. The complex has E3 ligase activity and ubiquitylates a number of proteins including the HIF-αs, atypical protein kinase Cλ (aPKCλ), deubiquitylating enzyme-1 (VDU-1), as well as two subunits of the RNA Polymerase II complex (RNAPII), Rpb1 and Rpb7. During normoxia, translated HIF-αs are hydroxylated on conserved proline residues located within L(XY)LAP motifs by the O₂, Fe(II) and oxyglutarateregulated Egl-9 type proline hydroxylases. This results in pVHL-dependent ubiquitylation and proteasomal degradation of HIF-αs. During hypoxia, proline hydroxylation is inhibited and HIF-αs are not ubiquitylated and degraded by the proteasome. Rather, they accumulate and regulate transcription of HIF-responsive genes, including angiogenic factors which contribute to tumor growth. Loss of pVHL function in hereditary VHL disease results in tumors which are highly vascularized, such as hemangioblastomas, angiomas and renal clear cell carcinomas (RCC). The VHL gene is also mutated in about 50-75% of sporadic RCC. A body of evidence indicates that accumulation of HIF and induction of HIF-gene products are necessary and sufficient for the growth of RCC tumors. However, tumors derived from genetically manipulated mouse embryonic stem cells VHL(−/−) (teratomas) or mouse embryonic fibroblasts (fibrosarcoma) are smaller compared to tumors derived from VHL(+/−) or VHL(+/+) cells. This implies that the effects of VHL may depend on the genetic background and, that VHL can be necessary for cell proliferation. The deletion of the VHL gene is embryonic lethal, while conditional knockout of VHL leads to hemangiomas and aberrant angiogenesis in multiple organs, but not to RCC. There is also a familial form of RCC that is not associated with any known inactivation of pVHL E3 ubiquitin ligase complex. Collectively, these findings indicate that mechanisms other than pVHL-dependent regulation of HIF-αs contribute to the pathogenesis of RCC.

Kidney cancer is a lethal malignancy with frequent occurrence in the adult population. Each year approximately 32,000 new cases are identified, and the incidence increases at a rate of about 2.5% per year (Linehan, W. M., and Zbar, B. (2004) Focus on kidney cancer. Cancer Cell 6, 223-228). The incidence is twofold higher in males than in females. Early identification of cancer exclusively confined to the kidney usually has a good prognosis, with 95% survival over 5 years. However, kidney cancer is usually asymptomatic until it metastasizes, at which time the survival rate is only about 18% over 2 years. Surgical removal remains the primary form of treatment. Thus, early detection and identification of markers that may serve to Identify individuals with a predisposition to this deadly disease is of crucial importance.

There is therefore a need for investigation of alternative mechanisms for the pathogenesis of RCC and similar carcinomas, which will provide more specific and more comprehensive therapeutic and pharmacological targets to diagnose, treat, and prognosticate with respect to such cancers.

### SUMMARY OF THE INVENTION

The present inventors investigated the hydroxylation of proline P1465 within the pVHL-binding domain of a novel pVHL target, the large subunit of RNA Polymerase II, Rpb1 and discovered that hydroxylation of P1465 phosphorylation of Ser-5 of Rpb1 CTD and ubiquitylation of Rpb1 are induced by oxidative stress in a VHL-dependent manner and do not lead to Rpb1 degradation. It was discovered that RCC cells having functional pVHL but expressing a P1465A Rpb1 mutant form tumors when injected into nude mice. It is clear that regulation of proline hydroxylation on Rpb1 is an important pathway controlling RCC tumorogenesis.

Accordingly, one embodiment of the present invention provides methods for diagnosing, treating and/or prognosing cancer in a patient. The method comprises detecting an amount, a localization, or a modification of a protein or enzyme implicated in the pVHL-dependent ubiquitylation pathway of DNA-bound Rpb1.

Another embodiment provides methods for determining an appropriate treatment regimen in a patient with cancer. The method relies on assessing tumor grade based on aggressiveness of a tumor, with a higher tumor grade corresponding to a more aggressive tumor. The methods comprise detecting at least one biomarker indicative of the tumor grade in a sample of the tumor.

Methods of inhibiting tumor growth are also provided. The methods comprise administering a pharmacological agent promoting hydroxylation of Rpb1, Ser-5 phosphorylation of Rpb1, or both.

A further embodiment is directed to methods for monitoring cancer development in an individual exhibiting a high risk factor that places the individual in a population at an increased risk for cancer development. The methods comprise first, qualitatively or quantitatively measuring a biomarker in the pVHL-dependent ubiquitylation of DNA-bound Rpb1 pathway in samples derived from a control population of cancer-free subjects. The number of subjects is statistically sufficient to establish a norm for the measurement in the population. Once a norm is generated, an individual's cancer status may be monitored by periodically testing the individual at risk by qualitatively or quantitatively measuring the biomarker in a sample derived from the individual and comparing it to the norm that was generated. If the individual's measurement deviates significantly from the norm, the individual may be referred for more expensive or invasive tests. The control group may comprise the general population, a general population selected for being cancer-free, or a specific population of subjects also exhibiting the high risk factor and selected for being cancer-free.

These and other embodiments and aspects of the invention will be further detailed and better understood by reference to the following figures, detailed description and examples therein.

### BRIEF DESCRIPTION OF THE DRAWINGS

FIG. 1. Illustrates that low grade oxidative stress induces VHL-dependent hydroxylation of P1465, phosphorylation of Ser-5 and ubiquitylation without causing Rpb1 degradation.

(A) Specificity of HP antibody detecting Rpb1 hydroxylated on P1465 (Rpb1-P(OH)). HP antibody detects hydroxylated Rpb1 in the total cellular (T) and nuclear (N) in absence of the competitor and in the presence of non-hydroxylated peptide competitor (NHPC) but not of the same amount of hydroxylated peptide competitor (HPC).
(B) Total nuclease digested cellular lysates from cells collected after H₂O₂ (25 μM) treatment at the indicated time points were analyzed by immunoblotting with HP antibody: Time point “0” indicates collection immediately at the end of 15 min exposure. High and low exposures of HP blots are shown. H14—detects Ser-5 phosphorylation. Note constitutive increase in Rpb1 Ser-5 phosphorylation in VHL(+) cells. Rb—blot probed with antibody against retinoblastoma protein to demonstrate equal protein loading; HA(VHL)—blot probed with anti-HA antibody detecting tagged VHL.
(C) P1465A mutation prevents hydroxylation, phosphorylation and ubiquitylation of Rpb1 in response to H₂O₂. Both wild type and P1465A Rpb1 were expressed at the similar protein level in 786-0 VHL(+) cells (compare lanes 1 and 3). Oxidative stress induces P1465 hydroxylation, Ser-5 phosphorylation and ubiquitylation of the His-tag Rpb1 wild type (right, lane 2), but not P1465A mutant (right, lane 4). Total nuclease digested lysates were immunoprecipitated with anti-Histidine antibody and blots were immunoblotted with the indicated antibodies.
(D) Oxidative stress induces binding of Ser-5 hyperphosphorylated. (H14, Rpb1-pS⁵, left) and P-1465-hydroxylated (HP, Rpb1-P(OH), right) Rpb1 to pVHL, as determined by co-immunoprecipitation with the anti-HA antibody from the nuclease digested total lysates.
(E) Proline 1465 is necessary for Rpb1-pVHL interaction in response to the oxidative stress. The wild type (lanes 3 and 4), but not the P1465A mutant (lanes 1 and 2) Rpb1, is co-immunoprecipitated with HA-pVHL (lanes 3 and 4). Oxidative stress induces strong ubiquitylation of Rpb1 co-immunoprecipitated with pVHL (HA antibody) as measured by immunoblotting with the anti-His antibody (lane 4) (left). Both Rpb1 Wt and P1465A mutant were expressed at approximately the same protein level (right).

FIG. 2. Illustrates that nuclear extracts from cells treated with H₂O₂ has increased hydroxylating activity towards Rpb1 but not HIF-1α peptide.

(A+B). H₂O₂ stimulates in vitro hydroxylation of Rpb1 peptide in the nuclear extracts from VHL(+) but not VHL(−) cells, as measured by binding of [³⁵S] labeled pVHL.
(C) Oxidative stress does not stimulate hydroxylation of HIF-α peptide in nuclear extracts from VHL(+) cells (compare lanes 4 and 5). Lane 1 in each panels shows percent input of [³⁵S] labeled pVHL; Lanes 2 and 3 show capture of [³⁵S] pVHL by hydroxylated and non-hydroxylated peptide, respectively; lane 4 and 5 show respectively capture of [³⁵S] pVHL by non-hydroxylated peptide hydroxylated in vitro in the extracts from indicated control cells (lane 4) or cells treated with H₂O₂ (lane 5).
(D) Quantification from indicated (n) number of hydroxylating experiments, using two, 24- and 37-amino acid long, Rpb1 peptides and a 17-amino acid long HIF-1α peptide.

FIG. 3. Illustrates that oxidative stress induced hydroxylation and Ser-5 phosphorylation occur on DNA-engaged Rpb1, and is accompanied by accumulation of all three HIF prolyl 4-hydroxylases and pVHL in the crude chromatin fraction.

(A+B) Immunoblot analysis of the nuclear pellet fractions from renal clear cell carcinoma 786-0 VHL(+) and (−) cells (A) or A-498 (13) collected 4 h after treatment with 25 μM H₂O₂. Rpb1-pS⁵-Rpb1 phosphorylated on Ser-5; D—fraction after DNase I/micrococcal nuclease digestion; 0.5 M and 1 M NaCl represent salt extractions of the nuclease digest. PHD1, 2 3-HIF prolyl-4-hydroxylases 1, 2 and 3.
(C) quantification of PHDs accumulation in combined crude chromatin fractions (D and both 0.5 M extracted fractions) in response to oxidative stress in 786-0 VHL(+) and (−) cells from 4 independent experiments.
(D) Rpb1-pS⁵ immunoprecipitated using H14 antibody from denatured crude chromatin extracts extracts is P-1465 hydroxylated (HP) and ubiquitylated in 786-0 VHL(+) (lanes 1 & 2) but not in VHL(−) (lanes 3 & 4) cells in response to H₂O₂ treatment.
(E) Deubiquitylation of Rpb1 (same fraction as shown in D, lane 2) by isopeptidase T and L3 (UCH, lane 3). Deubiquitylation is prevented by ubiquitin aldehyde (UbA, lane 4). Lane 1—input extract, lane 2—input extract incubated with UbA only.

FIG. 4. Shows that the knock-down of PHD1 inhibits, but the knock-down of PHD2 stimulates hydroxylation and Ser-5 phosphorylation of Rpb1 in response to oxidative stress. Blots of total cellular extract (TCE) or crude chromatin fraction (Chrom FR) were probed with the indicated antibodies.

(A) Stable knock-downs of PHD1 and PHD2 using TRC shRNA lentivirus constructs in 786-0 VHL(+) cells.
(B) Analysis of Rpb1 hydroxylation, Ser-5 phosphorylation in PHD1, 2 and 3 MEF knockouts.

FIG. 5. Illustrates that PHDs and pVHL form a multiprotein complex with Ser-5 phosphorylated and hydroxylated Rpb1 in chromatin extracts

FIG. 6. Illustrates that expression of Rpb1 mutant, P1465A, but not wild type cDNA in 3T3 fibroblasts induces formation of tumors.

(A) Comparison of expression of mRNA for endogenous mouse Rpb1 (mRpb1) with exogenous, human, flag-tagged Rpb1.
(B) A mean±SE of tumor size from 8 tumors formed by each clonal cell line expressing hRpb1 P1465A (left). Ultrasound photographs of the two representative subcutaneous tumors (right).
(C) 100× magnification of representative sections stained with hematoxylin & eosin (HE) and PCNA staining of representative sections from the indicated tumors (left); estimation of the human Rpb1 mRNA in the RNA extracted from five individual tumors at the time of final resection (T).

FIG. 7. Illustrates that expression of P1465A Rpb1 mutant but not the wild type cDNA in 786-0 VHL(+) cells partially restores tumor formation in nude mice.

(A) Expression of wild type and mutant Rpb1 in chromatin fractions from indicated clonal cell lines.
(B) A mean±SE of tumor size from the indicated number of tumors formed by each cell line. Numbers of tumors bigger or equal to 2 mm³ per numbers of injections are shown in parenthesis.
(C) Photographs of ultrasound pictures of the representative tumors. White arrows point to the tumor mass.
(D) 100× magnification of hematoxilin and eosin (HE) stained representative sections shows a large number of blood vessels and sinuses (arrows) in the tumor derived from VHL(−) cells. Only small capillaries were seen in the tumors from 786-0 VHL(+)/Rpb1P1465A tumors.

FIG. 8. Sets forth a Western blot analysis of chromatin enriched extracts from human tumors and matching normal kidneys immunoblotted for hydroxylated Rpb1. RCC —renal cell carcinoma; K-Kidney. Tumor 1 shows a major increase in hydroxylation of Rpb1. Tumor 2 shows a strong decrease in hydroxylation, and tumor 3 shows a very small increase, as compared to normal kidneys.

FIG. 9. Schematic model of the proposed regulation of the oncogenic process by P1465 hydroxylation and pVHL-dependent ubiquitylation of Rpb1. Hydroxylation and pVHL-dependent ubiquitylation of Rpb1 modulate the activity of RNAPII allowing for appropriate adaptation to oxidative stress. Inhibition of this pathway at any level affects RNAPII activity and physiological adaptation, leading to tumorigenesis. Simultaneous disruption of degradation of HIF-α results in its accumulation, stimulation of angiogenesis and tumor growth.

### DETAILED DESCRIPTION OF THE INVENTION

Renal clear cell carcinoma (RCC) is the most frequent and highly malignant cancer linked to the loss of activity of the von Hippel-Lindau tumor suppressor protein (pVHL). Epidemiologically, RCC is associated with cigarette smoking, hypertension, antihypertensive therapy, diabetes and obesity. In particular, a role for oxidative stress, including that resulting from cigarette smoking or changes in the blood flow through kidney has been proposed. pVHL is a component of a multiprotein complex containing elongins C and B, Cullin 2, and the RING-H2 finger protein Rbx-1. The complex has E3 ligase activity and ubiquitylates a number of proteins including the HIF-αs; atypical protein kinase C λ (aPKCλ); deubiquitylating enzyme-1 (VDU-1); and two subunits of the RNA Polymerase II complex (RNAPII), Rpb1 and Rpb7. During normoxia, translated HIF-αs are hydroxylated on conserved proline residues located within L(XY)LAP motifs by the O₂, Fe(II) and oxyglutarate-regulated Egl-9 type proline hydroxylases. This results in pVHL-dependent ubiquitylation and proteasomal degradation of HIF-αs. During hypoxia, proline hydroxylation is inhibited and HIF-αs are not ubiquitylated and degraded by the proteasome. Rather, they accumulate and regulate transcription of HIF-responsive genes, including angiogenic factors which contribute to tumor growth.

The VHL gene is mutated in about 50-75% of sporadic RCC. Loss of pVHL function in hereditary VHL disease results in highly vascularized RCC and also other capillary tumors, such as hemangioblastoma of central nervous system and retinal angioma (RCC). A body of evidence indicates that accumulation of HIF and induction of HIF-target gene products are necessary and sufficient for the growth of RCC tumors. However, several findings indicate that mechanisms other than pVHL-dependent regulation of HIF-αs contribute to the pathogenesis of RCC. For example, conditional knockout of VHL leads to hemangiomas and aberrant angiogenesis in multiple organs, but not to RCC. In addition, there is a familial form of RCC that is not associated with any known inactivation of pVHL E3 ubiquitin ligase complex. Lastly, tumors derived from genetically manipulated VHL(/) mouse embryonic stem cells (teratomas) or mouse embryonic fibroblasts (fibrosarcoma) are smaller compared to tumors derived respectively from VHL(+/−) or VHL(+/+) cells suggesting that in addition to its tumor suppressing activity pVHL may be necessary for cell proliferation.

The present inventors established that pVHL binds and ubiquitylates Rpb1, the large subunit of the RNA Polymerase II complex (RNAPII), hyperphosphorylated on Ser-5 residues of heptad repeats within C-terminal domain (CTD). pVHL binding and ubiquitylation occur through a LGQLAP motif that bears sequence and structural similarity to a pVHL-binding domain within HIF-1α and that is located in the pocket between Rpb1 and another subunit of the RNAPII complex, Rpb6, on the surface of the complex. This interaction of pVHL with Rpb1, similar to its interaction with HIF-α, requires hydroxylation of the proline P1465 within this binding motif.

Phosphorylation of multiple residues within 52 heptads of CTD has well recognized function in regulating RNAPII activity. In the preinitiation complex, CTD is hypophosphorylated. Upon transition from transcription initiation to elongation, the Ser-5 residues within multiple heptad repeats of CTD become phosphorylated. Subsequently, during processive elongation, Rpb1 becomes hyperphosphorylated on Ser-2 residues within the heptad repeats. Phosphorylation of the CTD is required for the RNAPII complex to associate with multiple protein factors, including cap and polyA binding proteins and splicing factors, which regulate posttranscriptional RNA processing. There is also growing evidence that RNAPII activity is regulated by ubiquitylation. Rpb1 is ubiquitylated and degraded in response to DNA lesions induced by UV light and high, millimolar concentrations of H₂O₂, possibly due to stalling of the RNAPII complexes. Stalled RNAPII prevents access of repair enzymes to DNA, which further increases the mutation rate, and prohibits other RNAPII complexes from following the stalled ones, thereby inhibiting transcription. Not surprisingly, stalled RNAPII is a potent inducer of apoptosis. Recent evidence indicates that dephosphorylation of Rpb1 is required for ubiquitylation leading to degradation. However, ubiquitylation of Rpb1 occurs also in its hyperphosphorylated form and during on-going transcription. Polyubiquitylation of Rpb1 during active transcription involves both lysine K48- and K63-linked ubiquitin chains. This is intriguing because ubiquitylation through both lysines has been reported to regulate protein activity without targeting them for proteasomal degradation. The laboratory of the present inventor previously demonstrated that pVHL regulates transcription elongation of the transcripts for tyrosine hydroxylase, the rate limiting enzyme in catecholamine synthesis. All together these data imply that pVHL-dependent ubiquitylation of Rpb1 modulates RNAPII activity and gene expression. Thus loss of such modulation could contribute to activation of oncogenic gene profiles and stimulation of oncogenesis.

The present invention is based on the discovery and confirmation by the inventors that pVHL-dependent modulation of Rpb1 function regulates oncogenesis in some carcinomas, and in particular in renal clear cell carcinoma. Specifically, low grade oxidative stress, a common pathophysiological factor in cancer, induces pVHL-dependent P1465 hydroxylation and polyubiquitylation of Rpb1 engaged on DNA without apparent Rpb1 degradation, as well as phosphorylation of Ser-5 within the CTD. Importantly, P1465 hydroxylation involves activity of EgIn 2 (PHD1) prolyl hydroxylase and occurs in a pVHL-dependent manner in chromatin fraction. It was also discovered that mutation of P1465 preventing hydroxylation and ubiquitylation of Rpb1 induces oncogenic potential in renal carcinoma cells expressing functional VHL, as well as in mouse 3T3 fibroblasts. These effects occur in the presence of functional pVHL and without HIF accumulation. Thus pVHL recognition of the hydroxylated proline can suppress tumorigenesis.

Rpb1 is the large subunit of the RNA Polymerase II complex (RNAPII). In mammals, its C-terminal domain (CTD) is composed of 52 heptad repeats, each containing multiple Sers and threonines that can undergo phosphorylation. In the preinitiation complex, CTD is hypophosphorylated. Upon transition from transcription initiation to elongation, the Ser-5 residues within multiple heptad repeats of CTD become phosphorylated. Subsequently, during processive elongation, Rpb1 becomes hyperphosphorylated on Ser-2 residues within the heptad repeats. Phosphorylation of the CTD is required for the RNAPII complex to associate with multiple protein factors, including cap and polyA binding proteins and splicing factors, which regulate posttranscriptional RNA processing.

To date, precise understanding of the role of Rpb1 ubiquitylation in the regulation of RNAPII activity is limited. Rpb1 is ubiquitylated and degraded in response to DNA lesions induced by UV light and high, millimolar concentrations of H₂O₂, possibly due to stalling of the RNAPII complexes. Stalled RNAPII prevents access of repair enzymes to DNA, which further increases the mutation rate, and prohibits other RNAPII complexes from following the stalled ones, thereby inhibiting transcription. Not surprisingly, stalled RNAPII is a potent inducer of apoptosis.

Recent evidence indicates that dephosphorylation of Rpb1 is required for ubiquitylation leading to degradation. However, ubiquitylation of Rpb1 occurs also in its hyperphposphorylated form and during on-going transcription. Ubiquitylation of Rpb1 during active transcription involves both lysine K48 and K63 on ubiquitin. This is intriguing because ubiquitylation through both lysines has been reported to regulate proteins activity without targeting them for proteasomal degradation. The present inventor established that pVHL binds and ubiquitylates Rpb1, specifically hyperphosphorylated on Ser-5 residues within the CTD, but not hypophosphorylated or phosphorylated on Ser-2 within CTD. pVHL binding and ubiquitylation occur through a LGQLAP motif on the surface of Rpb1 near the CTD that bears sequence and structural similarity to a corresponding domain within HIF-1α. This interaction of pVHL with Rpb1, similar to its interaction with HIF-1α, requires hydroxylation of the Proline 1465 within this binding motif.

Given these findings, the present inventor surmised that pVHL-dependent modulation of Rpb1 function regulates oncogenesis in renal cancer. Hydroxylation within Rpb1 was biochemically established and its role in RCC tumorigenesis was investigated. It was determined that low grade oxidative stress, a common pathophysiological factor in cancer, induces P1465 hydroxylation of Rpb1, which leads to pVHL-dependent polyubiquitylation of Rpb1 without apparent Rpb1 degradation. This implies that polyubiquitylation may Serve other functions in regulating the activity of RNAPII. It was further demonstrated that profound changes in cell proliferation and cell cycle result when mutation of P1465 prevents hydroxylation and ubiquitylation of Rpb1. These effects occur in the presence of functional pVHL and without HIF accumulation. This novel pathway for oncogenesis, in particular of renal tumors, provides several novel targets for cancer diagnosis, treatment and screening.

Accordingly, one embodiment of the invention is directed to methods of diagnosing, treating, and/or prognosing cancer in a patient. The method comprises detecting an amount, a localization, or a modification of a protein or enzyme implicated in the pVHL dependent ubiquitylation pathway of DNA-bound Rpb1. This non-degradative ubiquitylation pathway involves DNA-bound Rpb1 having an altered P1465 hydroxylation status and an altered Ser-5 hyperphosphorylation status. In specific embodiments, the enzyme comprises a proline-4 hydroxylase, and according to more specific embodiments, the proline-4-hydroxylase comprises proline hydroxylase 1 (PHD 1), PHD 2, or PHD 3 or some combination thereof.

In some embodiments, biomarkers may be utilized to detect the presence, amount, localization or modifications. In specific embodiments, the biomarker comprises detecting a decrease in amount of P1465 hydroxylation of Rpb1 or a decrease in amount of Ser-5 phosphorylation of Rpb1 or both. According to more specific embodiments, the biomarker is detected by using an antibody. The biomarker may, for example, comprise an altered P1465 hydroxylation status in Rpb1 or an altered C-terminal domain (CTD)—Ser-5 hyperphosphorylation status in Rpb1 or both. Specific antibodies useful to detect these biomarkers include HP for the P1465 hydroxylation status and H14 for the CTD Ser-5 hyperphosphorylation status.

Specific methods are directed to diagnosing and treating cancers such as carcinomas or sarcomas. Particular carcinomas include, for example, renal clear cell carcinoma, and particular sarcomas include, for example, sarcoma fibroblasts.

Another embodiment of the present invention is directed to methods for determining an appropriate treatment regimen-in a patient with cancer by assessing tumor grade. The tumor grade is based on aggressiveness of a tumor, wherein a more aggressive tumor is assigned a higher tumor grade. The method comprising detecting at least one biomarker indicative of the tumor grade in a sample of the tumor. The biomarker may comprise, for example, a nuclear pattern of prolyl hydroxylation of P1465 of Rpb1, a nuclear pattern of phosphorylation of CTD Ser-5, or a nuclear pattern of pVHL-dependent ubiquitylation of DNA-bound Rpb1. In specific embodiments, the biomarker comprises a pattern of subcellular distribution of one or more of prolyl-4 hydroxylase enzymes, PHD1, PHD2, and PHD3, or a post-translational modification of one or more of prolyl-4 hydroxylase enzymes, PHD1, PHD2, and PHD3. A pattern of subcellular distribution associated with aggressive tumors includes a decreased nuclear detection of PHD1 and 3 and an augmented nuclear accumulation of PHD2. Such a pattern may be assigned either a qualitative or quantitative value, relative to a pattern norm.

A further embodiment is directed to methods of inhibiting tumor growth. The methods comprise administering a pharmacological agent promoting hydroxylation of Rpb1, phosphorylation of Rpb1, or both. Promoting either of these enzymatic events typically results in pVHL ubiquitylation of DNA-bound Rpb1 and is correlated to suppression of tumor growth. In specific embodiments, a proline-4 hydroxylase agonist is administered, and in other specific embodiments, a Ser-5 kinase agonist is administered. In some embodiments, the tumor may be a carcinoma, for example, renal clear cell carcinoma or a sarcoma.

Another method embodiment is directed to methods for monitoring cancer development in an individual exhibiting a high risk factor that places the individual in a population at an increased risk for cancer development. In some embodiments, the high risk factor induces physiological oxidative stress in the individual. In specific embodiments the high risk factor comprises one or more of use of tobacco products, exposure to UV radiation, recurrent dehydration, presence of a sleep apnea disorder, presence of a medical condition that correlates positively with development of heart disease, and presence of a gene that correlates with development of a cancer, for example, a carcinoma. In very specific embodiments, the carcinoma comprises renal clear cell carcinoma and in other very specific embodiments, the medical condition correlating positively with development of heart disease includes one or more of obesity, high blood pressure, elevated Serum triglycerides, and elevated cholesterol.

Since the novel biomarkers may be detected in nuclear cells, blood samples, in particular, white blood cells, are ideal candidates for inexpensive yet highly accurate population screening. The methods involve qualitatively or quantitatively measuring a biomarker for proteins or enzymes implicated in the pVHL-dependent ubiquitylation of DNA-bound Rpb1 pathway in samples derived from a control population of cancer-free subjects, the number being statistically sufficient to establish a norm for the measurement in the population.

According to one specific embodiment, the control population is derived from the general population, selected for being cancer-free. In another specific embodiment, the control population is derived from a population of subjects exhibiting at least one high risk factor, for example obese subjects, or smoking subjects, or obese smoking subjects, but also selected for being cancer-free at the time of generating the norm measurements. The individual at risk is periodically tested by qualitatively or quantitatively measuring the biomarker in a sample from the individual and comparing it to the norm generated from the control group measurements. The individual may be referred for additional more expensive or intrusive testing if the individual's measurement deviates significantly from the norm.

In specific embodiments, the biomarker is a modification of enzymes and/or proteins in the pVHL-dependent ubiquitylation pathway of DNA-bound Rpb1. For example, the biomarker may comprise an alteration in P1456 hydroxylation of Rpb1 or an alteration in C-terminal domain Ser-5 phosphorylation of Rpb1 or both. Typically, follow-up testing may be indicated where the alteration comprises a decrease relative to the norm, or relative to a measurement or set of measurements derived from the individual when the individual was cancer-free. In very specific embodiments, the individual's past measurements may serve as a sufficient and very precise control from which to generate a norm. This method is a bit more labor-intensive with respect to any given individual, but may be particularly desirable in individuals beginning the monitoring process early in life who seek to be monitored throughout a time frame rather than those seeking a one or two-time screening analysis.

Typically, the biomarker is detected by using an antibody, and in very specific embodiments the antibody comprises a polyclonal antibody. In more specific embodiments, the antibody comprises HP or H14 or both.

The following examples are intended only to illustrate various embodiments and aspects of the present invention and should not be construed as limiting the scope of the invention as defined by the claims.

### EXAMPLES

For purposes of the Examples, the following antibodies were used: H14 (Research Diagnostics), C21 (Santa Cruz), anti-cullin2 (NeoMarkers), VHL (Ig32) (Pharmingen), HA (I2CA5, Boeringer Manheim), anti-Rbx1 (Zymed), antielongin C (Signal Transduction), HIFIa and H1F2a (Novus), anti-ubiquitin (StressGen), anti-histidine (Invitrogen). Antibody against hydroxylated proline within the Rpb1 peptide (HP) was custom-made for the inventor by Alpha Diagnostic, Inc. (San Antonio, Tex.). Secondary antibodies were obtained from Sigma, and fluorescent secondary antibodies were purchased from Molecular Probes (Alexa 633 and Alexa 488) or from Santa Cruz. Synthetic biotinylated peptides were made by Alpha Diagnostic, Inc. The Rpb1 construct was based on the construct pAT7h1 (Nguyen et al., 1996).

### Example 1

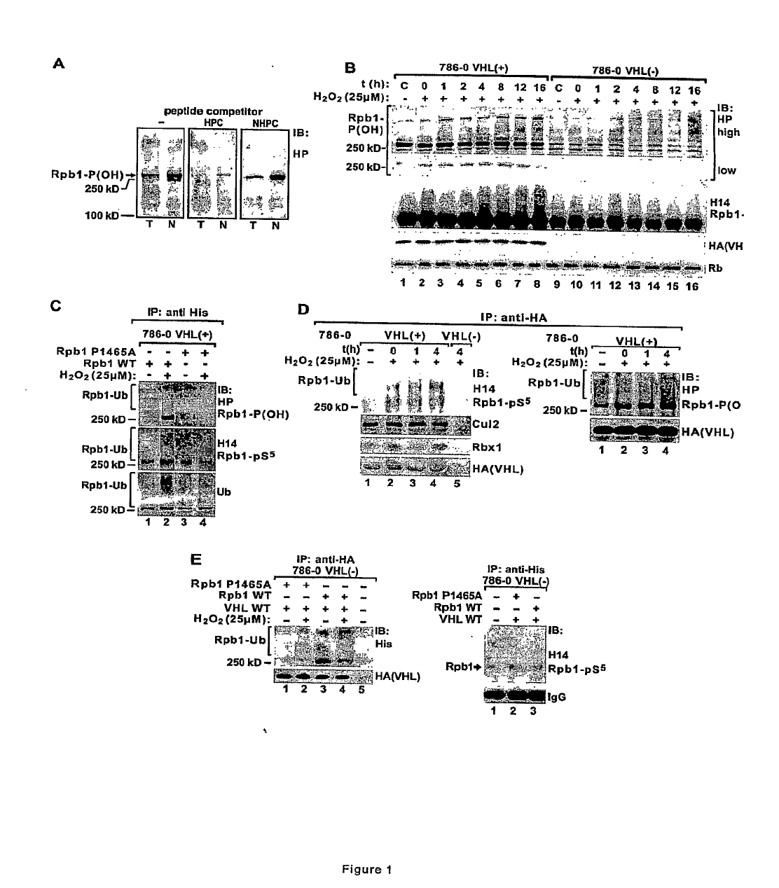

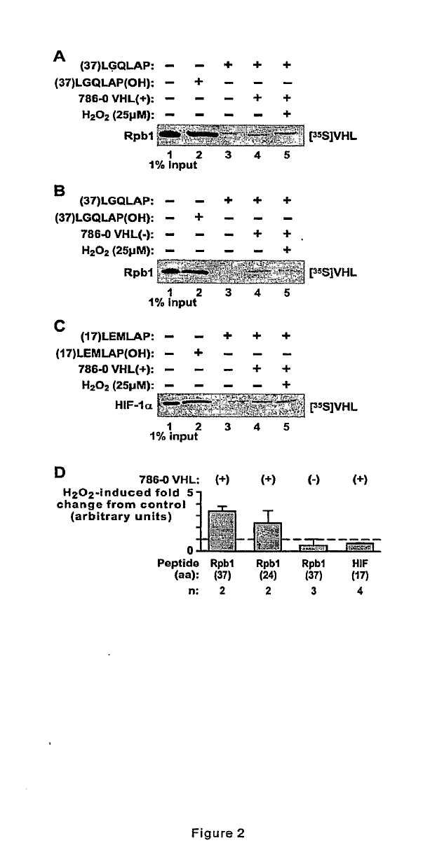

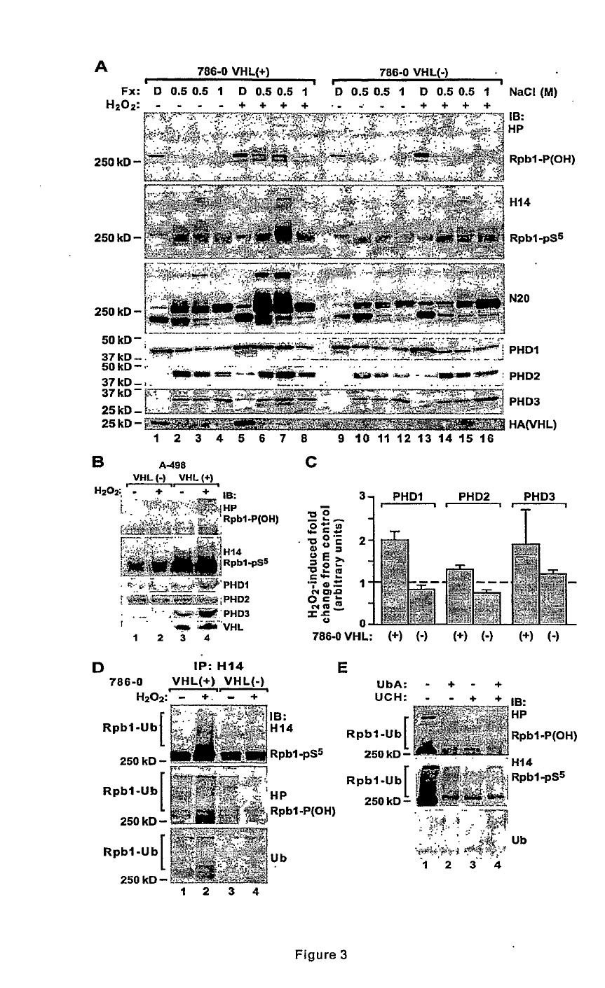

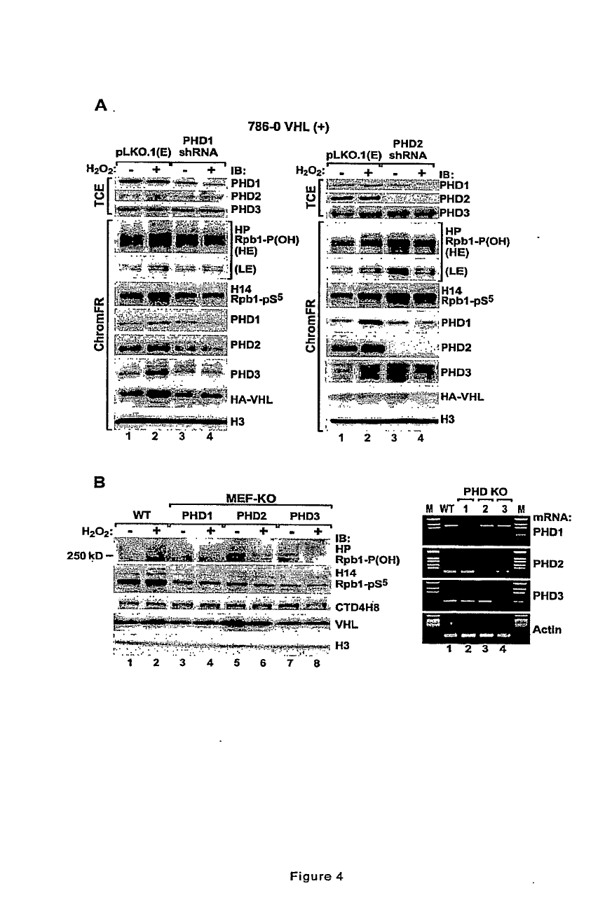

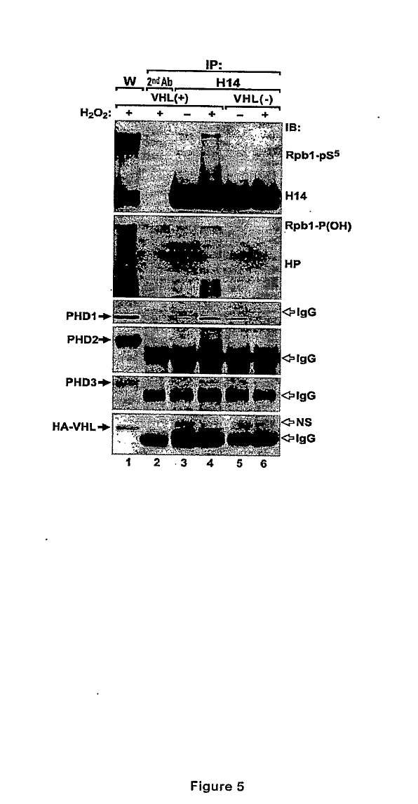

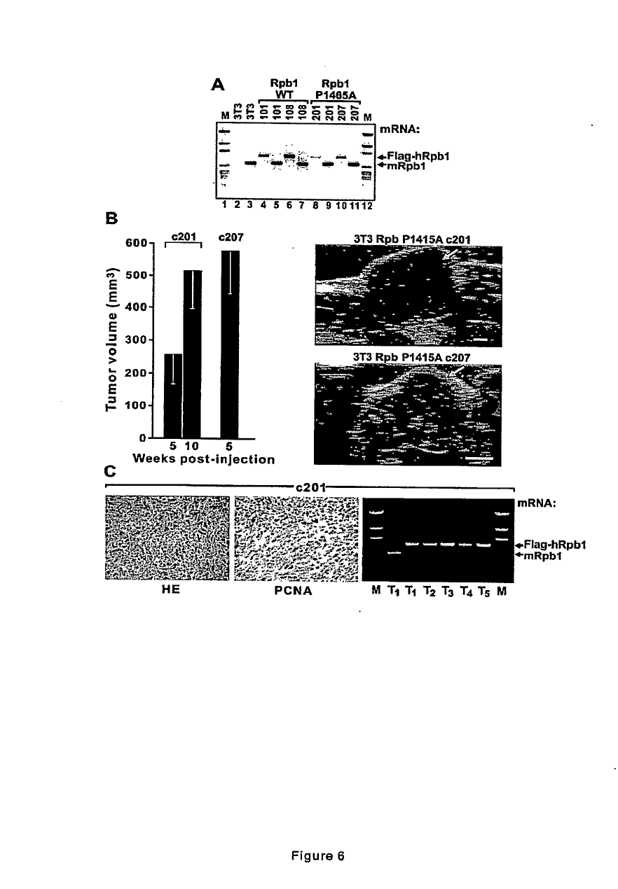

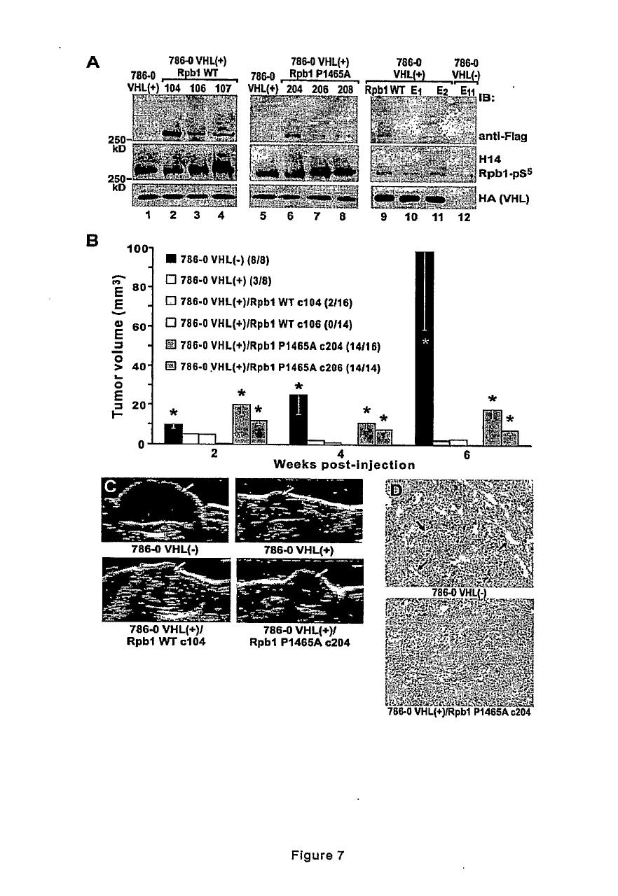

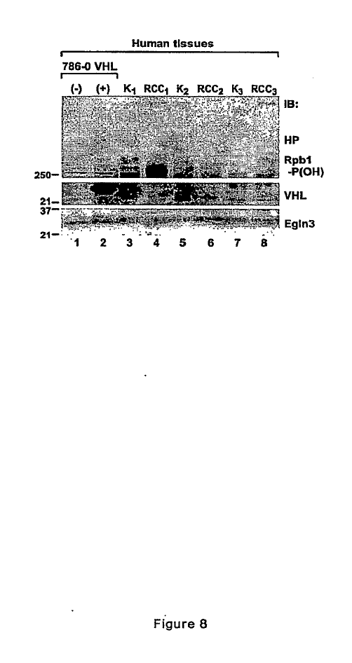

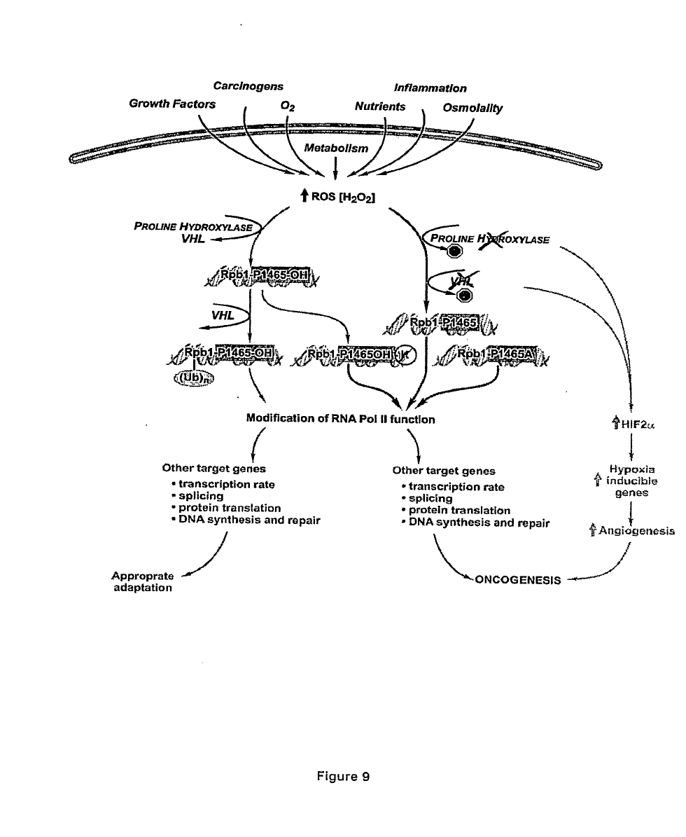

This Example illustrates the use of novel antibody to investigate P1465 hydroxylation and Rpb1 ubiquitylation. The antibody HP was developed against an Rpb1 peptide containing hydroxylated proline P1465 (FIG. 1A). The antibody was used to investigate the role of oxidative stress in P1465 hydroxylation and pVHL-dependent ubiquitylation of Rpb1. HP detects a major band at approximately 250 kD, which is highly enriched in the nuclear fraction as compared to total cellular extracts (FIG. 1A). The experiments were performed using RCC 786-0 cells, a well-characterized model of renal cell cancer, in which native pVHL is absent or stably reconstituted (in legend: 786-0 VHL(−) and VHL(+)). The cells were exposed briefly to a low concentration of H₂O₂ (25 μM, 15 min) to induce a generic oxidative stress, and assayed at the indicated time points after treatment.

It was found that a 15 min treatment with H₂O₂ induced persistent (lasting several hours) pVHL-dependent hydroxylation of Rpb1, and the appearance of a higher molecular weight ladder in extracts obtained by digestion with DNAse and micrococcal nuclease to enrich for Rpb1 engaged on the DNA (FIG. 1B). Interestingly, levels of Ser-5 phosphorylated Rpb1, both constitutive and H₂O₂ induced, were substantially higher in VHL(+) then in VHL(−) cells. These results were obtained in RCC 786-0 VHL(+) cells in which native pVHL is stably reconstituted in a cell line with a background nonfunctional deletion mutant of pVHL, 786-0 VHL(−) (17,18). A similar result was measured also in another RCC cell line, A-498 (FIG. 3B).

To further establish P1465 as the hydroxylation site of Rpb1, as well as the specificity of the HP antibody, a construct of histidine-tagged Rpb1 was transiently expressed, either wild type (WT) or P1465A mutant, in 786-0 cells (FIG. 1C). Both proteins were expressed at comparable levels under control conditions. Immunoprecipitation of the transfected Rpb1 using anti-histidine antibody demonstrated that H₂O₂ induced hydroxylation and ubiquitylation and Ser-5 phosphorylation of WT, but not P1465A mutant Rpb1 cells (FIG. 1C). The Rpb1 P1465A mutant showed some constitutive ubiquitylation that was not regulated by H₂O₂ (FIG. 1C, lanes 3 & 4).

The role of pVHL in ubiquitylation of Rpb1 was further supported the finding that treatment with H2O₂ stimulated rapid and sustained binding of pVHL to Ser-5 hyperphosphorylated (FIG. 1D, left) and hydroxylated (FIG. 1D right) Rpb1. This induction was detectable immediately following exposure (time point “0”), and persisted for at least 4 h (FIG. 1D). Co-immunoprecipitation of lysates from cells transfected with WT or P1465A histidine-tagged Rpb1 with an antibody against the hemagglutinin tag on pVHL showed that pVHL bound to histidine-tagged WT Rpb1 (FIG. 1E, lanes 3 and 4), but interacted only minimally with the P1465A mutant (FIG. 1E, lanes 1 and 2). Importantly, pVHL co-immunoprecipitated more of the higher molecular weight forms of Rpb1 in response to H₂O₂, (FIG. 1E, lane 4), indicating ubiquitylation of Rpb1. These data clearly show that oxidative stress induces the Rpb1-pVHL interaction.

The in vitro hydroxylation reactions of Rpb1 peptides by nuclear extracts from cells treated with hydrogen peroxide further confirm the detection of Rpb1 hydroxylation using the HP antibody (FIG. 2). They also further support the observation that oxidative stress-induced hydroxylation of Rpb1 requires the presence of wild type pVHL. The in vitro reactions of synthetic Rpb1 and HIF-1α peptides were performed using nuclear extracts from cells treated with 25 μM H₂O₂ and collected after 4 h. Incubation of the 37—(FIGS. 2A and 2D) or 24—amino acid long (FIG. 2D) Rpb1 peptides with extracts from H₂O₂-treated 786-0 VHL(+) cells, but not from 786-0 VHL(−) cells (FIGS. 2B and 2D), augmented peptide hydroxylation, as measured by the peptide's ability to capture [³⁵S]-labeled pVHL. In contrast, hydroxylation of the 17-amino acid long HIF-1α peptide in extracts from VHL(+) cells was not affected by H₂O₂ (FIGS. 2C and 2D). These data point out an important difference between the pathways leading to proline hydroxylation of Rpb1 and HIF-α in response to oxidative stress.

Rpb1 phosphorylated on Ser-5 is engaged on the DNA template in the promoter proximal region in the process of transitioning from transcription initiation to transcription elongation during active transcription. However, Rpb1 can be also phosphorylated on Ser-5 in its soluble, non-engaged form in the process inhibiting its transcriptional activity, because transcription initiation requires previous dephosphorylation to the hypophosphorylated Rpb1. In the experiments shown in FIG. 1 total cellular lysates that were digested with the nucleases to release bound Rpb1 were used in order to estimate steady-state levels or Rpb1 in the context of its potential degradation in response to oxidative stress. Because disappearance of phosphorylated and hydroxylated Rpb1 in response to the oxidative stress was not measured, rather, an actual increase in its accumulation (FIG. 1B), an attempt was undertaken to determine of hydroxylated Rpb1 was engaged on the DNA. In our experiments, we fractionated the pool of Rpb1 engaged on DNA using nuclease digestion and different salt extractions: the nuclear pellet remaining after standard extraction of nuclei with 0.3 M salt was digested with DNAse and micrococcal nuclease, and the digest was then extracted with the indicated concentrations of NaCl (FIG. 3). The high salt-extracted fractions did not contain DNA detectable by ethidium bromide staining on agarose gels, and were highly enriched in methylated histones (data not shown), thus representing a crude chromatin fraction. The HP antibody detected strong hydroxylation and the formation of higher molecular weight, ubiquitylated forms of Rpb1 in two chromatin fractions (0.5 M NaCl) from VHL(+) cells (FIG. 3A, lanes 6 and 7). This effect was consistently stronger in the second 0.5 M NaCl fraction. These modifications were absent in the corresponding fractions from VHL(−) cells (FIG. 3A, lanes 14 and 15). It is important to note that, in addition to the predominant hydroxylation and ubiquitylation of Rpb1 that require the presence of pVHL during oxidative stress, there is also some level of pVHL-independent Rpb1 hydroxylation. (FIG. 3A, lanes 1 & 5 and 9 &13). Two other antibodies against Rpb1 were also used: H14, and N20, which is specific for the N-terminal region of Rpb1, independent of its phosphorylation state. Similar to the HP antibody, these antibodies detected strong ubiquitylation of Rpb1 and its phosphorylation on Ser-5 in the high-salt chromatin fractions from VHL(+) cells in response to oxidative stress, but much less in VHL(−) cells. A similar results was found when we compared total crude chromatin fractions of A-498 VHL(+) and VHL(−) cells. Note the particularly strong effect of pVHL reconstitution stimulating the constitutive content of Ser-5 phosphorylated Rpb1 in the chromatin fraction from these cells (FIG. 3B).

### Example 2

This example demonstrates that hydroxylation and ubiquitylation occur on Ser-5 phosphorylated Rpb1.

Immunoprecipitation of the transfected histidine tagged wild type or P1465A mutant Rpb1 using anti-histidine antibody demonstrated that H₂O₂ induced hydroxylation and ubiquitylation and Ser-5 phosphorylation of WT, but not P1465A mutant Rpb1 cells (FIG. 1C). The Rpb1 P1465A mutant showed some constitutive ubiquitylation that was not regulated by H₂O₂ (FIG. 1C, lanes 3 & 4).

Rpb1 was immunoprecipitated using H14 antibody from the second 0.5 M chromatin fraction, and the immunoprecipitated Rpb1 was immunobloted with BP and anti-ubiquitin antibodies (FIG. 3D). Clearly, the Rpb1 hyperphosphorylated on Ser-5 is also hydroxylated and ubiquitylated in response to oxidative stress and this response depends on the presence of functional pVHL (FIG. 1C). To confirm ubiquitylation of the hydroxylated and hyperphosphorylated Rpb1, the extracts from the second 0.5 M chromatin fraction of VHL(+) cells were incubated with deubiquitylating enzymes, isopeptidase T and ubiquitin C-terminal hydrolase L3. This treatment results in disappearance of the high migrating forms (FIG. 3E. lane 3) which is prevented by ubiquitin aldehyde, an inhibitor of deubiquitylating enzymes (FIG. 3E, lane 4). This demonstrates that the higher migrating forms of Rpb1 generated in response to H₂O₂ result from polyubiquitylation.

### Example 3

This example demonstrates that hydroxylation of Rpb1 on P1465 occurs through activity of HIF-prolyl-4-hydroxylases and requires presence of pVHL. It also demonstrates that Ser-5 phosphorylation is dependent on hydroxylation of Rpb1 and the presence of pVHL.

Because of the similarity in the Rpb1 motif containing P1465 with an analogous motif on HIF-α, it was hypothesized that one or more of HIF prolyl hydroxylases hydroxylates Rpb1. PHDs represent a relatively novel family of deoxygenases utilizing Fe(II), ketoglutarate, molecular O₂ and ascorbate as cofactors, and were first identified as proline hydroxylases for HIF-αs (60). We discovered a presence and strict correlation of the levels of all three PHDs with the levels of Rpb1 hydroxylated in response to oxidative stress in the crude chromatin fractions of both 786-0 and A-498 cell lines expressing wild type pVHL, but not in those cells with non-functional pVHL (FIGS. 3A, 3B and 3C). The fractions of chromatin extract containing P1465-hydroxylated Rpb1 and stimulated levels of PHDs show also presence and enrichment in response to H₂O₂ of pVHL (3.5+/−1.1 fold increase). These data show that PHDIs1-3 are in physical proximity to Rpb1 and that their localization to the chromatin is regulated in parallel with P1465 hydroxylation of Rpb1, in a pVHL-dependent manner. Thus, they are likely to be prolyl hydroxylases for Rpb1. This conclusion is theoretically supported by the fact that both HIF-α and Rpb1 share the same LXXLAP sequence and structure in the region of proline hydroxylation.

In order to determine functional role of individual PHDs in hydroxylation of Rpb1 we performed knock-downs of individual PHDs using shRNA TRC lentiviral constructs (FIG. 4A). We also utilized MEFS from individual knock-out mice (FIG. 4B). The results consistently demonstrated that knock-down (FIG. 4A) or knock-out (FIG. 4B) of PHD1 abolished hydroxylation of Rpb1 in response to oxidative stress which was accompanied by the lack of induction of either of the three PHDs or pVHL in the chromatin fraction in response to oxidative stress. In contrast, the knock-down or knock-out of PHD2 had an opposite effect. It induced constitutive hydroxylation and ubiquitylation of Rpb1, and exposure of cells to oxidative stress was without further effect, or even decreased it. Interestingly there was a strong constitutive induction of PHD3, but not PHD1, and pVHL in crude chromatin fraction of PHD2 knock-down cells. Most interestingly Ser-5 phosphorylation followed closely the pattern of Rpb1 hydroxylation, i.e. knockdown of PHD1 decreased phosphorylation of Rpb1 on Ser-5 occurring in response to the oxidative stress, while knock-down of PHD2 strongly stimulated Ser-5 phosphorylation. Finally all three PHds and pVHL appear to be in the complex with Rpb1, induced by oxidative stress in crude chromatin fraction (FIG. 5). Altogether these data indicate that each of the three PHDs is involved in Rpb1 hydroxylation and Ser-5 phosphorylation but in a different mechanism. They also provide novel evidence that Rpb1 hydroxylation and Ser-5 phosphorylation are strongly interconnected.

### Example 4

This example demonstrates that Rpb1 mutant P1465A expressed in human renal epithelial cells or mouse fibroblasts stimulates tumor formation in nude mice.

Analysis of the proliferation rates of multiple clones of 786-0 VHL(+) cells expressing WT or P1465 A mutant Rpb1 reveals profound differences in their oncogenic properties. To determine proliferative effects of Rpb1 mutant, wild type and P1465A Rpb1 mutant were flag-tagged and stably expressed either in non transformed mouse 3T3 fibroblasts (FIG. 6) or in renal cancer cells, 786-0, with reconstituted pVHL so that they do not form tumors in mice (FIG. 7). FIG. 6A demonstrates expression of exogenous vs. endogenous Rpb1 in several clones of 3T3 fibroblasts. Clearly clones expressing P1465A mutant formed large and malignant tumors (FIG. 6B), but clones expressing wild type Rpb1 did not. Histological analysis revealed very strong staining for markers of cell proliferation (PCNA) and high number of blood vessels in those tumors (FIG. 6C). Clearly the expression of Rpb1 mutant was maintained in the tumors until the mice were sacrificed (FIG. 6C, right).

FIG. 7A shows expression of the flag-tagged Rpb1 in 786-) cells. Athymic mice were injected with 786-0VHL(−), 786-0VHL(+) cells, and two clones of 786-0 VHL(+) cells expressing either WT or P1465A Rpb1 (FIGS. 7 B and 7C). Within 2 weeks of injection, 786-0 VHL(+)/Rpb1PI46SA cells form tumors of approximately 10-20 mm³. At this point, tumors generated by 786-0 VHL(−) cells are of comparable size. Interestingly, starting at 4 weeks post-injection, tumors generated by 786-0VHL(−) cells begin to grow steadily, while tumors generated by VHL(+)PI465A cells plateau at 10-20 mm³ for the duration of the experiment (FIG. 7B). 786-0 VHL(+) and 786-0VHL(+)/Rpb1 WT cells either do not generate tumors, or in those few cases where tumors form, their sizes are barely measurable (less than 2 mm³) (FIGS. 7B and 7C). Histological evaluation of tumor sections after hematoxylin/eosin staining reveals that VHL(−) tumors have a large number of blood vessels, while VHL(+)/Rpb1PI465A tumors show very few blood vessels (FIG. 7D).

This finding suggests that the ability of VHL(−) tumors to continue growth after 4 weeks is due to HIF-induced angiogenesis; whereas, the early growth which is similar in VHL(−) and VHL(+)P1465A cells is independent of angiogenesis. VHL(+)/Rpb1P1465A tumors are malignant and contain round or oval shaped cells with abundant cytoplasm and large nuclei with open chromatin and evident nucleoli. Focal areas of necrosis and frequent mitotic figures are also identified. Importantly, tumors derived from VHL(+)/Rpb1PI465A, but not from VHL(−) cells show spindled features (FIG. 7D), indicating sarcomatoid differentiation and potential more aggressive behavior. Together, these data demonstrate that the P1465A Rpb1 mutant has oncogenic properties in the presence of the functional wild type pVHL in cells of epithelial and mesenchymal origin.

### Example 5

This example illustrates that human renal clear cell carcinomas have altered amounts of Rpb1 hydroxylated on P1465 and Ser-5 phosphorylated Rpb1.

Analysis of the initial sample of human RCC tumors as compared to the matched normal kidney counterpart shows that in some cases hydroxylation of Rpb1 is reduced in human tumors (FIG. 8). In addition in some cases we have observed reduction in Ser-5 phosphorylated Rpb1 (data not shown).

Here it was demonstrated that hydroxylation of P1465 of the large subunit of RNA Polymerase II hyperphosphorylated on Ser-5 and its subsequent pVHL-dependent ubiquitylation are an important tumor-suppressor mechanism preventing the growth of human renal cell carcinomas. Loss of P1465 hydroxylation is sufficient to overcome the tumor suppressor action of pVHL. This pathway represents a previously unrecognized control point for tumorigenesis in RCCs. In contrast to most known RCCs which involve mutation in pVHL disrupting its activity towards HIF, tumorigenesis via the pVHL/RNAPII pathway can occur in the context of a fully functional pVHL without HIF accumulation. The RCC tumors may result from the disruption of any step in this pathway. This can include a loss of pVHL function, or alternatively, loss of function of proteins other than pVHL, including mutations in Rpb1, hydroxylating enzymes, or other proteins necessary for hydroxylation and ubiquitylation of Rpb1. We propose the following mechanism for the pVHL-RNAPII pathway of tumor suppression (FIG. 9): hydroxylation of P1465 and subsequent pVHL-dependent ubiquitylation of Rpb1 modulate the activity of the RNAPII complex.

This regulatory pathway is active constitutively, and is further stimulated by low-grade oxidative stress and likely by other carcinogenic stressors. This mechanism causes optimal patterns of gene expression to assure healthy adaptations; for example, inhibition of mRNA synthesis in response to oxidative stress. Loss of P1465 hydroxylation and pVHL-mediated ubiquitylation disrupts the normal activity of RNAPII, causing abnormal patterns of gene expression which lead to increased cell proliferation, changes in cell cycle, tumor formation and loss of extracellular matrix. The finding that not only Rpb1 ubiquitylation, but also P1465 hydroxylation and even Ser-5 phosphorylation of Rpb1 within its fraction engaged on the DNA require presence of intact pVHL, represent a novel and important aspect of pVHL function. This could involve regulation of gene expression at the level of transition from transcription initiation to elongation, where phosphorylation of Ser-5 plays a major regulatory role. This can regulate transcription processivity or rate, or the ubiquitylation may affect co-transcriptional functions related to hyperphosphorylated Rpb1, such as binding of splicing factors or capping proteins.

Thus, ubiquitylation of Rpb1 may influence final mRNA products through changes in transcription as well as through changes in splicing, transport or translation of specific mRNAs at the post-transcriptional level. The molecular mechanisms by which the mutant P1465A exerts its action via Rpb1 are under investigation. It is intriguing that relatively low expression of the single amino acid mutant on a high endogenous Rpb1 background is sufficient for gain-of-function effects on cell growth. Interestingly, tumors derived from cells expressing the P1465A mutant contain many spindle-shaped cells, indicative of transformation to higher grade malignancies. Moreover, these malignant tumors show less angiogenesis than pVHL(−) derived tumors, consistent with the finding that the Rpb1 P1465A mutant does not stimulate HIF-2α, accumulation. The oxidative stress is a precipitating factor in many cancers, including renal cancer. Clearly, RNAPII activity in response to oxidative stress is regulated differently by the presence or absence of pVHL-dependent ubiquitylation. Loss of components of Rpb1 hydroxylation represent a potential mechanism by which reactive oxygen species could stimulate oncogenic processes, even in the presence of pVHL.

Similar to HIF-α P1465 hydroxylation is a prerequisite for pVHL-mediated ubiquitylation of Rpb1. Unlike HIF-α, we did not detect any apparent degradation of Rpb1 in response to hydroxylation/ubiquitylation. This conclusion is based on the observation that ubiquitylation in response to low grade oxidative stress is easily detectable without proteasomal blockers, and there is not only no detectable decrease, but actually an increase, in the amount of Rpb1 engaged on DNA for up to several hours after treatment with H₂O₂. An increase in the half-life of engaged Rpb1 phosphorylated at Ser-5 after translation was also measured and was inhibited with cycloheximide (data not shown). These results confirm that ubiquitylation of Rpb1 in VHL(+) cells does not lead to increased degradation, but instead seems to stabilize Rpb1.

These conclusions differ from the previously suggested role of pVHL-mediated polyubiquitylation in the degradation of Rpb1 as an adaptation to DNA damage. Thus the pVHL-mediated ubiquitylation of Rpb1 may have different regulatory functions towards Rpb1, both leading to its degradation and preventing its degradation. They are likely to be tissue-specific and to depend on the type or level of stressor and activation of different signaling pathways. It is becoming increasingly evident that protein ubiquitylation, in addition to mediating targeting for proteasomal degradation, can have a number of non-canonical activities in cell function. Importantly, ubiquitylation of Rpb1 during active transcription involves not only K48 but also K63 on ubiquitin. Polyubiquitylation through K63 on ubiquitin is not inhibitory for protein activity but may in some conditions stimulate it. Ubiquitylation through K48 regulates transcription also independently from proteolysis. Clearly, low grade oxidative stress induces prolylhydroxylation and pVHL-dependent ubiquitylation. However, low level of pVHL independent ubiquitylation in VHL(−) cells in response to oxidative stress was also measured. Others reported that extremely high doses of H202 cause comparable ubiquitylation of Rpb1 in VHL(−) and VHL(+) cells.

Collectively these data imply that Rpb1 is regulated by ubiquitylation in several, potentially not mutually exclusive, ways. The role of pVHL in protein ubiquitylation is also complex. Not only does it directly target proteins for ubiquitylation, but it also ubiquitylates and targets for degradation the deubiquitylating enzymes, a process which in a secondary manner may affect ubiquitylation of some currently unidentified substrates. Recent evidence has demonstrated that pVHL can induce assembly of polyubiquitin chains to the HIF-1 a substrate, not only through ubiquitin K48 but also through other lysines. This strongly supports the that the role of pVHL in protein ubiquitylation may extend beyond targeting for proteasomal degradation.

The similarity between proline hydroxylation motifs within HIF-α and Rpb1 suggests that the Egln group of proline hydroxylases participate in the regulation of both proteins. The data confirm this prediction, and indeed, using functional approaches, show that that PHD 1 is Rpb1 hydroxylase. While elucidation of the role of PHD3 requires further experiments, the knock down or knockout of PHD2, the main PHD hydroxylating HIF-a has an opposite effect. Thus this PHD has an inhibitory effect on the constitutive Rpb1 hydroxylation. The reason for this is currently not understood It is known however that PHDs form complexes with one another. Thus, perhaps PHD2 may sequester PHD1 and or PHD3 away from Rpb1 under control conditions, inhibiting constitutive hydroxylation.

The present work addresses an important question about the broader role of pVHL and HIF in tumorigenesis and cell growth. While the loss of pVHL is necessary and sufficient for the optimal growth of RCC tumors due to the stimulation of HIF activity and HIF targets (Kondo et al., 2002; Kondo et al, 2003), our results show that the deactivation of pVHL can play an oncogenic role, in the absence of HIF accumulation and of HIF-induced vasculogenesis. A disassociation between HIF accumulation and pVHL-dependent tumor formation has been described in the case of type 2C VHL disease, where certain mutations within VHL gene, such as V84L and LI 88V, cause pheochromocytoma tumors without promoting RCC. Moreover, certain non-epithelial tumors derived from embryonic stem cells or mouse embryonic fibroblasts are smaller in homozygous VHL—than in heterozygous VHL—cells, due to HIF-mediated growth arrest (Mack et al., 2005). This implies that while pVHL has tumor suppressing activity in epithelial cells, it can be also necessary for cell growth in other cell types.

The present invention is based on the work establishing that pVHL-dependent hydroxylation and ubiquitylation of Rpb1 play a major role in control of renal cancer oncogenesis. This newly discovered pathway can occur independent of pVHL-dependent regulation of renal oncogenesis through HIFs and provides mechanisms for novel interventions and methods for treating, diagnosing and screening cancers.

What is claimed:

1. A method of diagnosing, treating, and/or prognosing cancer in a patient, the method comprising detecting an amount, a localization, or a modification of a protein or enzyme implicated in the pVHL dependent ubiquitylation pathway of DNA-bound Rpb1 .

2. The method according to claim 1, wherein detecting comprises detecting a biomarker for the amount, localization or modification.

3. The method according to claim 1, wherein the DNA-bound Rpb1 has an altered P1465 hydroxylation status and an altered Ser-5 hyperphosphorylation status.

4. The method according to claim 2, wherein the biomarker comprises detecting a decrease in amount of P 1465 hydroxylation of Rpb1 or a decrease in amount of Ser-5 phosphorylation of Rpb1 or both.

5. The method according to claim 1 wherein the enzyme comprises a proline-4 hydroxylase.

6. The method according to claim 5, wherein the proline-4-hydroxylase comprises proline hydroxylase 1 (PHD 1), PHD 2, or PHD 3 or some combination thereof.

7. The method according to claim 2, wherein detecting a biomarker comprises detecting the biomarker with an antibody.

8. The method according to claim 7, wherein the biomarker comprises an altered P1465 hydroxylation status in Rpb1 or an altered C-terminal domain (CTD)—Ser-5 hyperphosphorylation status in Rpb1 or both.

9. The method according to claim 8, wherein the antibody comprises HP for the P1465 hydroxylation status and H14 for the CTD Ser-5 hyperphosphorylation status.

10. The method according to claim 1 wherein the cancer comprises a carcinoma or a sarcoma.

11. The method according to claim 10, wherein the carcinoma comprises renal clear cell carcinoma.

12. A method for determining an appropriate treatment regimen in a patient with cancer by assessing tumor grade based on aggressiveness of a tumor, the method comprising detecting at least one biomarker indicative of the tumor grade in a sample of the tumor.

13. The method according to claim 12, wherein the biomarker comprises a nuclear pattern of prolyl hydroxylation of P 1465 of Rpb1, a nuclear pattern of phosphorylation of CTD Ser-5, or a nuclear pattern of pVHL-dependent ubiquitylation of DNA-bound Rpb1 .

14. The method according to claim 12, wherein the biomarker comprises a pattern of subcellular distribution of one or more of prolyl-4 hydroxylase enzymes, PHD1, PHD2, and PHD3.

15. The method according to claim 12, wherein the biomarker comprises a post-translational modification of one or more of prolyl-4 hydroxylase enzymes, PHD1, PHD2, and PHD3.

16. The method according to claim 14, wherein the pattern comprises a decreased nuclear detection of PHD1 and 3 and an augmented nuclear accumulation of PHD2, indicative of an increase in tumor grade.

17. A method of inhibiting tumor growth comprising administering a pharmacological agent promoting hydroxylation of Rpb1, phosphorylation of Rpb1, or both.

18. The method of claim 17, wherein promoting results in pVHL ubiquitylation of DNA-bound Rpb1.

19. The method of claim 17, wherein promoting comprises administration of a proline-4 hydroxylase agonist.

20. The method of claim 17, wherein promoting comprises administration of a Ser-5 kinase agonist.

21. The method of claim 17, wherein the tumor comprises a carcinoma or a sarcoma.

22. The method of claim 21, wherein the tumor comprises a carcinoma and the carcinoma comprises renal clear cell carcinoma.

23. A method for monitoring cancer development in an individual exhibiting a high risk factor that places the individual in a population at an increased risk for cancer development, the method comprising:
a. qualitatively or quantitatively measuring a biomarker in the pVHL-dependent ubiquitylation of DNA-bound Rpb1 pathway in samples derived from a control population of cancer-free subjects, the number being statistically sufficient to establish a norm for the measurement in the population;
b. periodically testing the individual at risk by qualitatively or quantitatively measuring the biomarker in a sample derived from the individual and comparing it to the norm generated in (a); and
c. referring the individual for further testing if the measurement demonstrates a significant deviation from the norm generated in (a).

24. The method according to claim 23, wherein the control population of cancer free subjects is selected from a general population of cancer free subjects.

25. The method according to claim 23, wherein the control population of cancer free subjects is selected from a population of cancer free subjects exhibiting the high risk factor.

26. The method according to claim 23, wherein the sample comprises blood.

27. The method according to claim 23, wherein the sample comprises white blood cells.

28. The method according to claim 23, wherein the biomarker is a modification of enzymes and/or proteins in the pVHL-dependent ubiquitylation pathway of DNA-bound Rpb1.

29. The method according to claim 23, wherein the biomarker is an alteration in P1456 hydroxylation of Rpb1 or an alteration in C-terminal domain Ser-5 phosphorylation of Rpb1 or both.

30. The method according to claim 29, wherein the alteration comprises a decrease.

31. The method according to claim 23, wherein the biomarker is detected with an antibody.

32. The method according to claim 31, wherein the antibody comprises a polyclonal antibody.

33. The method according to claim 32, wherein the antibody comprises HP or H14 or both.

34. The method according to claim 23, wherein the high risk factor induces physiological oxidative stress in the individual.

35. The method according to claim 23, wherein the high risk factor comprises one or more of use of tobacco products, exposure to UV radiation, recurrent dehydration, presence of a sleep apnea disorder, presence of a medical condition that correlates positively with development of heart disease, and presence of a gene that correlates with development of a cancer.

36. The method according to claim 35, wherein the high risk factor comprises-presence of a gene that correlates with development of a cancer and the cancer comprises a carcinoma.

37. The method according to claim 36, wherein the carcinoma comprises renal clear cell carcinoma.

38. The method according to claim 35, wherein the medical condition comprises one or more of obesity, high blood pressure, elevated serum triglycerides, and elevated cholesterol.
